(19) United States
(12) Patent Application Publication
(10) Pub. No.: US 2025/0271815 A1
(43) Pub. Date: Aug. 28, 2025

(54) CROWN ASSEMBLY FOR AN ELECTRONIC WATCH

(71) Applicant: Apple Inc., Cupertino, CA (US)

(72) Inventors: Steven C. Roach, San Francisco, CA (US); Sameer Pandya, Sunnyvale, CA (US); Erik G. de Jong, San Francisco, CA (US); Erik L. Wang, Redwood City, CA (US); Peter Kardassakis, Mountain View, CA (US); Steven P. Cardinali, Campbell, CA (US)

(21) Appl. No.: 19/207,710

(22) Filed: May 14, 2025

Related U.S. Application Data
(63) Continuation of application No. 18/492,110, filed on Oct. 23, 2023, now Pat. No. 12,326,697.
Application No. 18/492,110 is a continuation of application No. 17/507,589, filed on Oct. 21, 2021, now Pat. No. 11,796,968.
Application No. 17/507,589 is a continuation of application No. 16/215,607, filed on Dec. 10, 2018, now Pat. No. 11,194,298.
(60) Provisional application No. 62/725,239, filed on Aug. 30, 2018.

(51) Int. Cl. G04G 21/08; G01D 5/30; G04B 37/10; G04G 9/00; G04G 17/08; G04G 21/02

(52) U.S. Cl. CPC G04G 21/08; G01D 5/30; G04G 9/007; G04G 17/08; G04G 21/025; G04B 37/10

(57) ABSTRACT

An electronic watch may include a housing defining a side wall having a through-hole and a crown assembly including an actuation member. The actuation member may include a crown shaft extending through the through-hole and having an exterior portion defining an input surface and a crown ring coupled to the exterior portion of the crown shaft and electrically isolated from the crown shaft. The crown assembly may further include an optical encoder component attached to the actuation member and defining a group of optical features. The electronic watch may further include an optical detector configured to detect rotation of the crown assembly by detecting motion of the group of optical features and an electrocardiogramansor comprising a sensing component. The sensing component may be conductively coupled to the actuation member via a conductive path at least partially defined by the crown shaft.

### CROSS-REFERENCE TO RELATED APPLICATIONS

This application is a continuation of and claims the benefit of priority to U.S. patent application Ser. No. 18/492,110, filed Oct. 23, 2023, and titled “Crown Assembly for an Electronic Watch,” which is a continuation of U.S. patent application Ser. No. 17/507,589, filed Oct. 21, 2021, and titled “Crown Assembly for an Electronic Watch,” which issued as U.S. Pat. No. 11,796,968 on Oct. 24, 2023, which is a continuation of U.S. patent application Ser. No. 16/215,607, filed Dec. 10, 2018, and titled “Crown Assembly for an Electronic Watch,” which issued as U.S. Pat. No. 11,194,298 on Dec. 7, 2021, which claims the benefit under 35 U.S.C. § 119 of U.S. Provisional Patent Application No. 62/725,239, filed Aug. 30, 2018, and titled “Crown Assembly for an Electronic Watch,” the contents of which are incorporated herein by reference in their entireties.

### FIELD

The described embodiments relate generally to electronic devices, and more particularly to a crown for a wearable electronic device.

### BACKGROUND

Electronic devices frequently use physical input devices to facilitate user interaction. For example, buttons, keys, dials, and the like can be physically manipulated by users to control operations of the device. Physical input devices may use various types of sensing mechanisms to translate the physical manipulation to signals usable by the electronic device. For example, buttons and keys may use collapsible dome switches to detect presses, while dials and other rotating input devices may use encoders or resolvers to detect rotational movements.

### SUMMARY

An electronic watch may include a housing defining a side wall having a through-hole and a crown assembly including an actuation member. The actuation member may include a crown shaft extending through the through-hole and having an exterior portion defining an input surface and a crown ring coupled to the exterior portion of the crown shaft and electrically isolated from the crown shaft. The crown assembly may further include an optical encoder component attached to the actuation member and defining a group of optical features. The electronic watch may further include an optical detector configured to detect rotation of the crown assembly by detecting motion of the group of optical features and an electrocardiograph sensor comprising a sensing component. The sensing component may be conductively coupled to the actuation member via a conductive path at least partially defined by the crown shaft.

The electronic watch may further include a switch configured to be actuated by the crown assembly in response to an axial input applied to the crown assembly, a friction guard having a shear plate positioned between the switch and the optical encoder component and conductively coupled to the optical encoder component, and a bracket comprising a body structure and a conductor coupled to the body structure. The conductive path may be further defined by the optical encoder component and the conductor and may be configured to carry electrical signals from a user in contact with the input surface to the electrocardiogramansor. The body structure may include a polymer material, and the conductor may be at least partially encapsulated by the polymer material.

The electronic watch may further include a switch configured to be actuated by the crown assembly in response to an axial force applied to the crown assembly, and a friction guard at least partially positioned between the switch and the optical encoder component and conductively coupled to the optical encoder component. The axial force may be transferred from the crown assembly to the switch through the friction guard, and the conductive path may be further defined by the friction guard. The crown assembly may be rotatable relative to the housing, the crown assembly may rotate against the friction guard when the crown assembly is rotated, and the friction guard may protect the switch from rotational friction from the crown assembly.

The electronic watch may further include, within an internal volume of the housing, a bracket, a frame coupled to the bracket and attached to the housing, and a wear plate positioned between an inner surface of the frame and configured to electrically isolate the optical encoder component from the frame.

The electronic watch may further include a display positioned at least partially within the housing and configured to display a graphical output, a transparent cover coupled to the housing, and a touch sensor positioned below the transparent cover and configured to detect touch inputs applied to the transparent cover, and the electronic watch may be configured to modify the graphical output in accordance with a detected rotation of the crown assembly.

An electronic watch may include a housing at least partially defining an internal volume, biometric sensing circuitry within the internal volume, and a crown assembly configured to rotate and translate relative to the housing. The crown assembly may include a crown shaft defining an axial end surface, a crown ring coupled to the crown shaft and defining a series of tactile features arranged around a circumference of the crown ring, and an optical encoder component attached to the crown shaft and defining a group of optical features. The crown shaft and the optical encoder component may define a conductive path configured to conductively couple the crown assembly to the biometric sensing circuitry. The biometric sensing circuitry may be part of an electrocardiograph sensor.

The electronic watch may further include a switch and a friction guard having a shear plate positioned between the crown assembly and the switch and configured to transfer axial forces from the crown assembly to the switch. The electronic watch may further include a bracket positioned in the internal volume and comprising a metal flange, a polymer body structure, and a conductor coupled to the polymer body structure and electrically isolated from the metal flange by the polymer body structure. The friction guard may be in contact with the conductor, and the conductive path may be further defined by the friction guard and the conductor. The electronic watch may further include a conductive lubricant at an interface between the crown assembly and the friction guard.

The optical encoder component may be attached to the crown shaft via a threaded connection. The axial end surface of the crown shaft may define an input surface for the biometric sensing circuitry.

An electronic device may include a housing at least partially defining an internal volume, an electrocardiograph sensor within the internal volume and comprising a sensing component, a frame within the internal volume and attached to the housing, and a bracket attached to the frame. The bracket may include a body structure and a conductor at least partially encapsulated in the body structure. The electronic device may also include a crown assembly having an external portion positioned outside of the internal volume and configured to rotate and translate relative to the housing. The bracket and the frame may at least partially enclose a portion of the crown assembly, and the crown assembly and the conductor may define a conductive path configured to conductively couple the sensing component to an object in contact with the external portion of the crown assembly.

The electronic device may further include a band coupled to the housing, a display at least partially within the internal volume of the housing, a transparent cover coupled to the housing and positioned such that the display is visible through the transparent cover, and a touch sensor at least partially within the internal volume and configured to detect touch inputs applied to the transparent cover.

The crown assembly may include a crown shaft having an internal portion within the internal volume and an optical encoder component attached to the internal portion of the crown shaft and defining a group of optical features. The electronic device may further include an optical detector configured to detect rotation of the crown assembly by detecting motion of the group of optical features, and the optical encoder component may define part of the conductive path.

The electronic device may include a friction guard in contact with the bracket and the crown assembly and a switch positioned between the friction guard and the bracket and configured to be actuated in response to axial translation of the crown assembly. The friction guard may include a shear plate and a support leg configured to allow the shear plate to deflect relative to the bracket. The friction guard may be a single piece of conductive material.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 depicts an example wearable electronic device;

FIG. 2 depicts a schematic cross-sectional view of a portion of a wearable electronic device;

FIG. 3A depicts a portion of an input system for a wearable electronic device;

FIG. 3B depicts an exploded view of an input system for a wearable electronic device;

FIG. 3C depicts a component of the input system of FIG. 3B;

FIG. 4 depicts a cross-sectional view of an input system for a wearable electronic device; and

FIG. 5 depicts example components of a wearable electronic device.

### DETAILED DESCRIPTION

Reference will now be made in detail to representative embodiments illustrated in the accompanying drawings. It should be understood that the following description is not intended to limit the embodiments to one preferred embodiment. To the contrary, it is intended to cover alternatives, modifications, and equivalents as can be included within the spirit and scope of the described embodiments as defined by the appended claims.

The embodiments herein are generally directed to a crown of a wearable electronic device, such as an electronic watch (also referred to as a “smart watch” or simply a “watch”), and more particularly to a crown that acts as a contact point for a sensor, such as a biometric sensor, of the watch. For example, a smart watch may include a heart rate sensor, an electrocardiograph sensor, a thermometer, a photoplethysmograph sensor, a fingerprint sensor, or the like, all of which are examples of biometric sensors that measure or detect some aspect of a user's body. Such sensors may require direct contact with the user's body, such as via a finger. Accordingly, a device may include an external button, window, electrode, or other input feature or surface that a user may touch in order to allow the sensor to take a reading or measurement.

In some cases, the portion of the sensor that a user contacts to provide input to the sensor may be integrated with another input system of the watch. For example, as described herein, a watch may include a crown that accepts one or more types of inputs, such as rotational inputs (e.g., twisting, turning, or spinning the crown) and/or translational inputs (e.g., pressing axially on the crown). Such inputs may allow the user to provide inputs to the watch, such as to interact with a graphical user interface (e.g., by modifying a graphical output of a display in accordance with a direction of rotation of the crown), setting the time, or the like. In some cases, the crown itself, or a portion thereof, may also define an input surface for providing inputs to biometric sensing circuitry of a biometric sensor. This may provide several advantages. For example, because the crown is a familiar input mechanism for a watch, users will be familiar with providing inputs using the crown. Further, assuming the user has provided appropriate permissions, biometric sensing may be performed while the user is providing other types of inputs to the device.

In some cases, a biometric sensor may need to conductively couple to a user's body in order to function. For example, a heart rate monitor, electrocardiograph sensor, fingerprint sensor, or the like may need to conductively couple to the user's body (e.g., via a finger) in order to measure or detect the user's heart rate, heart rhythm, or other biometric information or data. Accordingly, in order to provide input to biometric sensing circuitry via a crown, a conductive path may be formed through the crown and into the watch housing. In this way, the user may simply contact the crown (or a portion thereof) in order to provide an input to the biometric sensor. Forming a conductive path through the crown may be difficult, however, because crowns may include numerous intricate components to facilitate the detection of rotational and axial inputs, and because it may be necessary or desirable to electrically isolate the crown so that it is not conductively coupled to the housing or another grounded component (which may reduce or destroy the effectiveness of the crown as an input feature.

As described herein, a conductive path may be formed through multiple components of a crown input system, while preventing the crown from being grounded by a housing or internal structure of the electronic watch. For example, a rotatable component of a crown may be formed of a conductive material. An end of the rotatable component, such as the end of a shaft, may contact a friction guard or other physical shield within the watch. In some cases, as described herein, the rotatable component is coupled to or includes an optical encoder component (e.g., a component of an optical encoder), such as a barrel that defines reflective features and also is formed of or includes a conductive material. Where an optical component is included, the optical component may contact the friction guard instead of or in addition to the shaft. The friction guard may prevent friction from the rotating component from damaging other internal components of the input system (such as a switch). The friction guard may also be formed of or include a conductive material. Because the rotatable component and the friction guard are in direct contact with one another and are each formed from or include conducive materials, these components may define at least part of a conductive path through the crown input system. This conductive path may conductively couple the user's finger to biometric sensing circuitry of a biometric sensor (while also isolating the conductive path from other conductive components such as a housing), thus allowing the crown to act as an input member not only for rotational and translational inputs, but also for the biometric sensor. Accordingly, the crown (or portions thereof) may be considered part of a biometric sensor, along with biometric sensing circuitry and/or other components.

As described herein, a crown input system may include structural components such as brackets, shrouds, frames, and the like, which may be formed of or include conductive materials and which may be secured to other conductive components, such as a metal housing. These components may form an internal enclosure that at least partially encloses or surrounds a rotatable component of the crown input system. In order to provide a conductive path that is not grounded to or otherwise conductively coupled to components that would have a deleterious effect on the operation of the biometric sensor, the crown input system may provide a conductive path through the structural components. For example, a conductor may be at least partially encapsulated in a polymer material of a bracket and provide an internal contact area (e.g., within the internal enclosure) that contacts the friction guard, and an external contact area (e.g., accessible via an outer surface of the internal enclosure) that is coupled to biometric sensing circuitry. The polymer material may electrically insulate or isolate the conductor so that the conductive path through the rotatable component and the friction guard can be passed through the enclosure without being grounded to the housing of the watch.

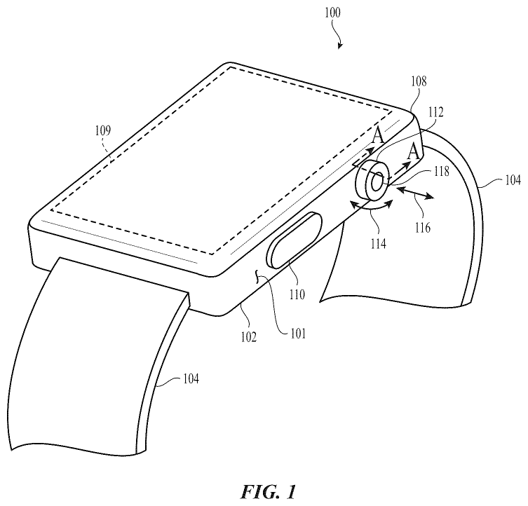

FIG. 1 depicts an electronic device 100. The electronic device 100 is depicted as a watch, though this is merely one example embodiment of an electronic device, and the concepts discussed herein may apply equally or by analogy to other electronic devices, including mobile phones (e.g., smartphones), tablet computers, notebook computers, head-mounted displays, digital media players (e.g., mp3 players), or the like.

The electronic device 100 includes a housing 102 and a band 104 coupled to the housing. The housing 102 may at least partially define an internal volume in which components of the device 100 may be positioned. The band 104 may be configured to attach the electronic device 100 to a user, such as to the user's arm or wrist.

The electronic device 100 also includes a transparent cover 108 coupled to the housing 102. The cover 108 may define a front face of the electronic device 100. For example, in some cases, the cover 108 defines substantially the entire front face and/or front surface of the electronic device. The cover 108 may also define an input surface of the device 100. For example, as described herein, the device 100 may include touch and/or force sensors that detect inputs applied to the cover 108. The cover may be formed from or include glass, sapphire, a polymer, a dielectric, or any other suitable material.

The cover 108 may overlie at least part of a display 109 that is positioned at least partially within the internal volume of the housing 102. The display 109 may define an output region in which graphical outputs are displayed. Graphical outputs may include graphical user interfaces, user interface elements (e.g., buttons, sliders, etc.), text, lists, photographs, videos, or the like. The display 109 may include a liquid crystal display (LCD), organic light emitting diode display (OLED), or any other suitable components or display technologies.

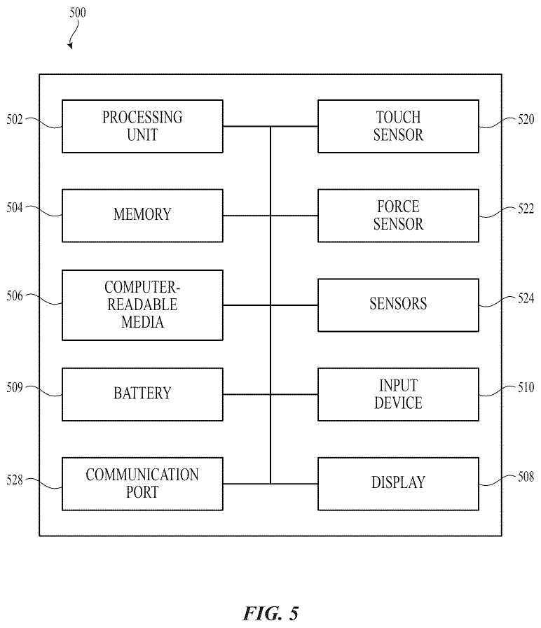

The display 109 may include or be associated with touch sensors and/or force sensors that extend along the output region of the display and which may use any suitable sensing elements and/or sensing systems and/or techniques. Using touch sensors, the device 100 may detect touch inputs applied to the cover 108, including detecting locations of touch inputs, motions of touch inputs (e.g., the speed, direction, or other parameters of a gesture applied to the cover 108), or the like. Using force sensors, the device 100 may detect amounts or magnitudes of force associated with touch events applied to the cover 108. The touch and/or force sensors may detect various types of user inputs to control or modify the operation of the device, including taps, swipes, multi-finger inputs, single- or multi-finger touch gestures, presses, and the like. Further, as described herein, the touch and/or force sensors may detect motion of an object (e.g., a user's finger) as it is interacting with a crown 112 of the electronic device 100. Touch and/or force sensors usable with wearable electronic devices, such as the device 100, are described herein with respect to FIG. 5.

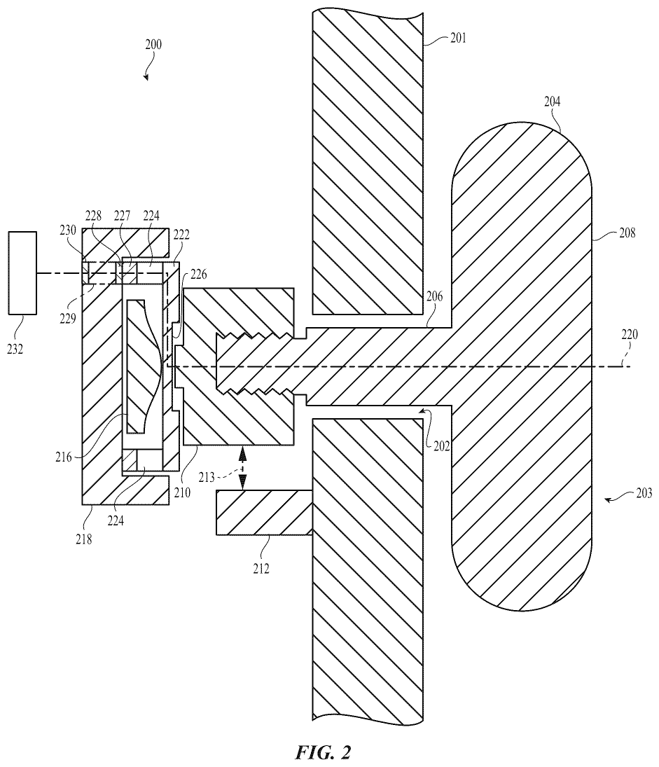

The electronic device 100 also includes a crown 112 (also referred to herein as a crown assembly) having a knob, external portion, or component(s) or feature(s) positioned along a side wall 101 of the housing 102. At least a portion of the crown 112 (e.g., a knob 208, FIG. 2) may protrude from the housing 102, and may define a generally circular shape or a circular exterior surface. The exterior surface of the crown 112 (or a portion thereof) may be textured, knurled, grooved, or may otherwise have features that may improve the tactile feel of the crown 112 and/or facilitate rotation sensing. At least a portion of the exterior surface of the crown 112 may also be conductively coupled to biometric sensing circuitry (or circuitry of another sensor that uses a conductive path to an exterior surface), as described herein.

The crown 112 may facilitate a variety of potential user interactions. For example, the crown 112 may be rotated by a user (e.g., the crown may receive rotational inputs). The arrow 114 in FIG. 1 illustrates example direction(s) of rotational inputs to the crown 112. Rotational inputs to the crown 112 may zoom, scroll, rotate, or otherwise manipulate a user interface or other object displayed on the display 109 (among other possible functions). The crown 112 may also be translated or pressed (e.g., axially) by the user, as indicated by arrow 116. Translational or axial inputs may select highlighted objects or icons, cause a user interface to return to a previous menu or display, or activate or deactivate functions (among other possible functions). In some cases, the device 100 may sense touch inputs or gestures applied to the crown 112, such as a finger sliding along a surface of the crown 112 (which may occur when the crown 112 is configured to not rotate relative to the housing 102) or a finger touching an end face of the crown 112. In such cases, sliding gestures may cause operations similar to the rotational inputs, and touches on an end face may cause operations similar to the translational inputs. As used herein, rotational inputs may include both rotational movements of the crown (e.g., where the crown is free to rotate), as well as sliding inputs that are produced when a user slides a finger or object along the surface of a crown in a manner that resembles a rotation (e.g., where the crown is fixed and/or does not freely rotate).

The crown 112 may also include or define an input feature 118 that facilitates input to biometric sensing circuitry or other sensing circuitry within the device 100. The input feature 118 may be a conductive surface that is conductively coupled, via one or more components of the device 100, to the biometric sensing circuitry. In some cases, the input feature 118 and/or the component(s) that define the input feature 118 are electrically isolated from other components of the device 100. For example, the input feature 118 may be electrically isolated from the housing 102. In this way, the conductive path from the input feature 118 to the biometric sensing circuitry may be isolated from other components that may otherwise reduce the effectiveness of the biometric sensor.

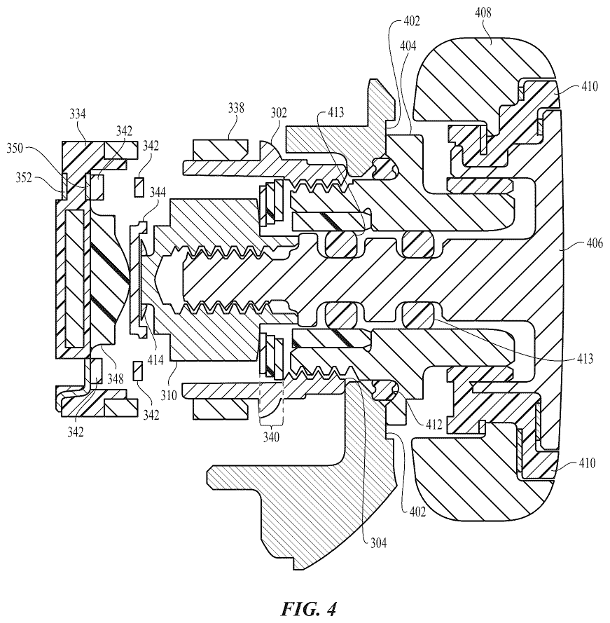

The input feature 118 may be an exterior surface of a component of a crown assembly, such as an exterior portion of a crown shaft (e.g., an exterior surface of the crown shaft 406, FIG. 4). In order to provide an input to the biometric sensor, a user may place a finger or other body part on the input feature 118. The biometric sensor may be configured to take a reading or measurement in response to detecting that the user has placed a finger or other body part on the input feature 118. In some cases, the biometric sensor may only take a reading or measurement when a sensing function is separately initiated by a user (e.g., by activating the function via a graphical user interface). In other cases, a reading or measurement is taken any time the user contacts the input feature 118 (e.g., to provide a rotational or translational input to the crown 112). The user may have full control over when the biometric sensor takes measurements or readings, and may even have the option to turn off the biometric sensing functionality entirely.

The device 100 may also include one or more haptic actuators that are configured to produce a tactile output through the crown 112. For example, the haptic actuator may be coupled to the crown 112 and may be configured to impart a force to the crown 112. The force may cause the crown 112 to move (e.g., to oscillate or vibrate translationally and/or rotationally, or to otherwise move to produce a tactile output), which may be detectable by a user when the user is contacting the crown 112. The haptic actuator may produce tactile output by moving the crown 112 in any suitable way. For example, the crown 112 (or a component thereof) may be rotated (e.g., rotated in a single direction, rotationally oscillated, or the like), translated (e.g., moved along a single axis), or pivoted (e.g., rocked about a pivot point). In other cases, the haptic actuator may produce tactile outputs using other techniques, such as by imparting a force to the housing 102 (e.g., to produce an oscillation, vibration, impulse, or other motion), which may be perceptible to a user through the crown 112 and/or through other surfaces of the device 100, such as the cover 108, the housing 102, or the like. Any suitable type of haptic actuator and/or technique for producing tactile output may be used to produce these or other types of tactile outputs, including electrostatics, piezoelectric actuators, oscillating or rotating masses, ultrasonic actuators, reluctance force actuators, voice coil motors, Lorentz force actuators, or the like.

Tactile outputs may be used for various purposes. For example, tactile outputs may be produced when a user presses the crown 112 (e.g., applies an axial force to the crown 112) to indicate that the device 100 has registered the press as an input to the device 100. As another example, tactile outputs may be used to provide feedback when the device 100 detects a rotation of the crown 112 or a gesture being applied to the crown 112. For example, a tactile output may produce a repetitive “click” sensation as the user rotates the crown 112 or applies a gesture to the crown 112. Tactile outputs may be used for other purposes as well.

The electronic device 100 may also include other inputs, switches, buttons, or the like. For example, the electronic device 100 includes a button 110. The button 110 may be a movable button (as depicted) or a touch-sensitive region of the housing 102. The button 110 may control various aspects of the electronic device 100. For example, the button 110 may be used to select icons, items, or other objects displayed on the display 109, to activate or deactivate functions (e.g., to silence an alarm or alert), or the like.

FIG. 2 depicts a partial cross-sectional view of an example electronic device 200 having an input system in which a conductive path 220 is defined through a crown assembly 203, as well as other components of the input system. For example, the conductive path 220 extends through the crown assembly 203, through a friction guard 222, through a bracket 218 via an embedded conductor 229, and to biometric sensing circuitry 232.

The input system shown in FIG. 2 uses the friction guard 222 to define part of the conductive path 220 while also allowing the crown assembly 203 to rotate and translate to accept rotational and translational inputs. The friction guard 222 also protects a switch 216 (which may detect axial inputs) from damage due to the rotation and translation of the crown. Thus, the crown input system defines a sufficiently isolated electrical path through the crown input system while also facilitating numerous other input functions and providing a robust and durable system.

The device 200 may be an embodiment of the device 100, and may include the same or similar components and may provide the same or similar functions as the device 100. Accordingly, details of the device 100 described above may apply to the device 200, and for brevity will not be repeated here.

As used herein, an “input system” may refer to and/or include mechanisms, systems, components, circuitry, and/or other components that together provide one or more input functions to a device. For example, the input system shown and described with respect to FIG. 2 (which may also be referred to as a crown input system) may include components such as an actuation member 204, an optical detector 212, a bracket 218, biometric sensing circuitry 232, an optical encoder component 210, a switch 216, a friction guard 222, and the like.

The device 200 includes a housing that defines a side wall 201 (e.g., which may correspond to the side wall 101, FIG. 1). The side wall 201 defines a through-hole 202, and an actuation member 204 may extend through the through-hole 202. The actuation member 204 may be a component of a crown assembly that extends through the housing and has a portion that is inside the housing and another portion that is outside the housing. The actuation member 204 defines a crown shaft 206 (at least a portion of which is inside the housing) and a knob 208 (at least a portion of which defines an exterior portion of the shaft that is outside the housing). As shown, the crown shaft 206 and the knob 208 may be formed as a unitary structure, though other actuation members may have different components and/or configurations, and may be defined by several different components that are attached together. The actuation member 204 may be formed from or include a conductive material (e.g., metal, carbon fiber, conductive polymer, conductive ceramics, or the like). Accordingly, an exterior surface of the actuation member 204 (e.g., the portion of the actuation member 204 that is outside of the device housing and is contactable by a user), and optionally other surfaces of the actuation member 204, form an input surface for a biometric sensor. More particularly, the electrical conductivity of the exterior surface facilitates a conductive path from a user's finger in contact with the actuation member 204.

The crown assembly 203 also includes an optical encoder component 210 (e.g., a barrel, sleeve, cylinder, or other component) attached to the actuation member 204. The optical encoder component 210 may be fixed to the actuation member 204 in such a way that the optical encoder component 210 moves with the actuation member 204. More particularly, when the actuation member 204 is rotated, the optical encoder component 210 rotates as well, and when the actuation member 204 is translated (e.g., axially, as a result of an axial input force), the optical encoder component 210 translates as well. While the crown assembly 203 shows the optical encoder component 210 as a barrel-shaped component, a different type or configuration of optical encoder component may be used, such as a sleeve, a cylinder, a nut, a splined component, a group of reflective members defining multiple facets, or the like.

The optical encoder component 210 may be a component of an optical encoder that determines one or more properties of a rotational input applied to the crown assembly 203. For example, the optical encoder component 210 define a group of optical features along an outer surface of the optical encoder component 210, and an optical detector 212 (which may also be considered a component of the optical encoder) may be configured to detect rotation of the crown assembly 203 by detecting motion of the group of optical features (via an optical path represented by arrow 213). In some cases, the optical detector 212 may include or may be associated with a light source or optical emitter that directs light (e.g., infrared light, laser light, visible light, or any other suitable radiation type) onto the outer surface of the optical encoder component 210, which may reflect the light onto the optical detector 212. The optical detector 212 may determine a property of motion of the crown assembly 203 based on the detected reflected light, such as a speed and/or direction of the motion of the crown assembly 203.

The optical features on the optical encoder component 210 may be any suitable features, such as grooves (e.g., parallel to the axis of the crown shaft 206), prisms, concave features, facets, threads, scratches, splines, recesses, or the like. The optical features may be arranged in an evenly spaced pattern around the optical encoder component 210 (e.g., the features may be separated by substantially a same distance). The regular arrangement may aid in the operation of the optical detector 212 and allow the optical detector 212 to determine properties of the motion and/or position of the crown assembly 203 (e.g., speed, direction, position, acceleration, or the like). In some cases, the optical features are not arranged in an evenly spaced pattern, but instead are arranged in an irregular pattern, a random pattern, or the like.

The optical encoder component 210 may be attached to the actuation member 204 in any suitable way, including a threaded connection (as shown in FIG. 2), adhesive, mechanical interlock structures, fasteners, pins, or the like. The optical encoder component 210 may be conductively coupled to the actuation member 204 to further define the conductive path 220 that conductively couples a user to a biometric (or other) sensing component 232. The optical encoder component 210 may be conductively coupled to the actuation member 204 via the threads, as shown, and/or via other conductive couplings (e.g., a wire or other conductive component that is conductively coupled to both the optical encoder component 210 and the actuation member 204).

In some cases the optical encoder component 210 may be omitted. In such cases the actuation member 204 itself may include optical features such as those described above as being on the optical encoder component 210, and the optical detector 212 may determine properties of the motion and/or position of the crown assembly 203 by directly sensing or detecting the optical features on the actuation member 204.

As noted above, the crown assembly 203 (which includes the actuation member 204 and the optical encoder component 210) may be translatable along an axis of the crown assembly 203 to provide an input to the device 200. In order to detect the axial input, the device 200 may include a switch 216 positioned between a bracket 218 and an end of the crown assembly 203. The switch 216 may be a dome switch, which may provide electrical switching functionality (e.g., closing a circuit upon actuation by the crown assembly 203) as well as a tactile output that may be felt or otherwise perceived by the user. For example, the user may feel a click, detent, or other sensation upon the collapse of the dome switch, thus indicating to the user that an input has been successfully provided to the device 200. In some cases, other types of switches or force sensing components may be used to detect axial inputs, which may be positioned similarly to the switch 216 in FIG. 2.

When the crown assembly 203 is rotated to provide a rotational input, the end of the crown assembly 203 that applies a force to actuate the switch 216 may slide, scrape, or shear against surfaces that support or are otherwise in contact with the crown assembly 203. If the crown assembly 203 were in contact directly with the switch 216, for example, an end of the optical encoder component 210 would apply a shearing or rotational friction on the surface of the switch 216 (due to the optical encoder component 210 rotating against the stationary switch 216). This friction may cause damage to the switch 216 and/or the optical encoder component 210. In some cases a friction guard 222 (or a portion thereof) is positioned between the switch 216 and the optical encoder component 210 (or between the switch 216 and the actuation member 204 in implementations where the optical encoder component 210 is omitted). The portion of the friction guard 222 that is between the optical encoder component 210 and the switch 216 (or between the actuation member 204 and the switch 216 in implementations where the optical encoder component 210 is omitted) is referred to as a shear plate 226.

Axial forces from the crown assembly 203 may be transferred to the switch 216 through the friction guard 222. For example, the friction guard 222 may be flexible so that the portion of the friction guard 222 that is between the optical encoder component 210 and the switch 216 can translate in response to an axial force applied by the crown assembly 203 and apply a corresponding force onto the switch 216 to actuate the switch 216. Because the friction guard 222 does not rotate, however, any friction due to rotation of the crown assembly 203 is applied to the friction guard 222 and not to the switch 216. This may help extend the life of the switch 216 by preventing the friction from damaging the outer surface of the switch 216, for example.

In order to support the friction guard 222 and maintain it in a desired position, the friction guard 222 may define support legs 224 that contact the bracket 218 and maintain the shear plate 226 in a desired position. In some cases, the support legs 224 are flexible and act as springs that allow the shear plate 226 to deflect (relative to the bracket 218) in response to an axial force applied to the crown assembly, and then return the shear plate 226 to a neutral or rest position after the axial force is removed. In some cases, the support legs 224 may bias the shear plate 226 against the end of the optical encoder component 210. This may help maintain physical contact with the optical encoder component 210, which may help reduce wear on the shear plate 226 and the optical encoder component 210.

The friction guard 222 may also define part of the conductive path 220 that may conductively couple the user's finger to a sensing component 232 through the crown assembly 203. Accordingly, the friction guard 222 may be formed of or include a conductive material, such as metal, metal alloy, carbon fiber, conductive polymers, or the like. In some cases, the friction guard 222 is a single piece of metal that defines the shear plate 226 and the support legs 224. One or more of the support legs 224 may define a contact portion 227 that is conductively coupled to another conductor 229 in the bracket 218, as described herein. The spring function of the support legs 224 may help maintain physical contact between the contact portion 227 and a corresponding contact portion 228 of the conductor 229, as well as between the shear plate 226 and the optical encoder component 210. Accordingly, the conductive path 220 is maintained during both rotational and axial movements of the crown assembly 203, allowing the sensing component 232 to take readings and/or measurements despite incidental or intentional movements of the crown assembly 203. As described herein, in order to reduce electrical noise introduced into the conductive path 220 when the crown assembly 203 is rotated, a conductive lubricant (e.g., a conductive grease) may be applied at the interface between the end of the optical encoder component 210 and the shear plate 226.

The bracket 218 may define a structural support for one or more components of the device 200. For example, the bracket 218 may be attached to the housing to provide a stable mounting structure for the switch 216 and the friction guard 222. The bracket 218 may also include a conductor 229, which may define a contact portion 228 (which is in contact with the support leg 224 of the friction guard 222) and a contact portion 230 that is accessible via the outside of the bracket 218. The contact portion 230 may be conductively coupled to the sensing component 232 (e.g., by a conductive trace, wire, flexible circuit component, or the like), thus defining part of the conductive path 220 between the crown assembly 203 and the sensing component 232. The conductor 229 may be a piece of metal that is coupled to a polymer body structure 234 of the bracket 218. In some cases the conductor 229 is at least partially encapsulated in the body structure 234 (e.g., by insert molding).

As shown in FIG. 2, components such as the bracket 218, the optical encoder component 210, the friction guard 222, a portion of the actuation member 204, and the switch 216 may be positioned within the internal volume of the device. Another portion of the actuation member 204, such as the knob 208, may be positioned outside of the internal volume (e.g., it may be an external portion of the actuation member 204 that is on the side of the device and is accessible to a user).

The input system described with respect to FIG. 2 may be used to carry electrical signals from a user to a sensing component 232. The sensing component 232 may be a component of any suitable type of sensor, such as a heart rate sensor, electrocardiograph sensor, temperature sensor, or the like. In some cases, the sensing component 232 may be coupled to or associated with other components that together form a sensor or sensing system, such as a processor, circuitry, a power source, or the like.

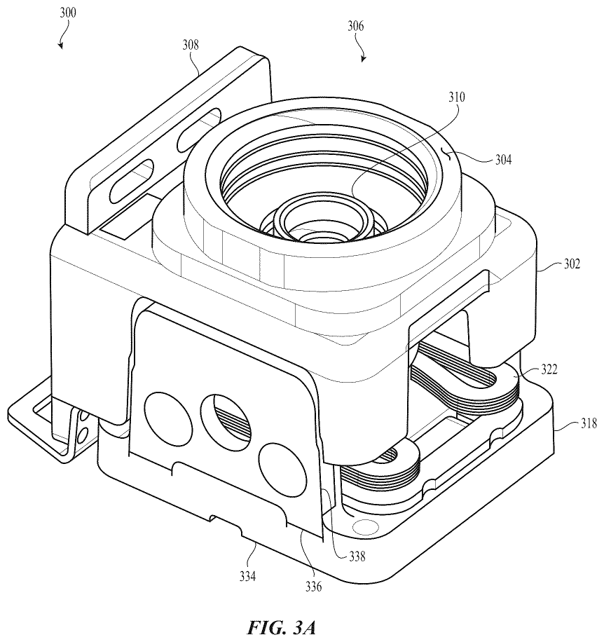

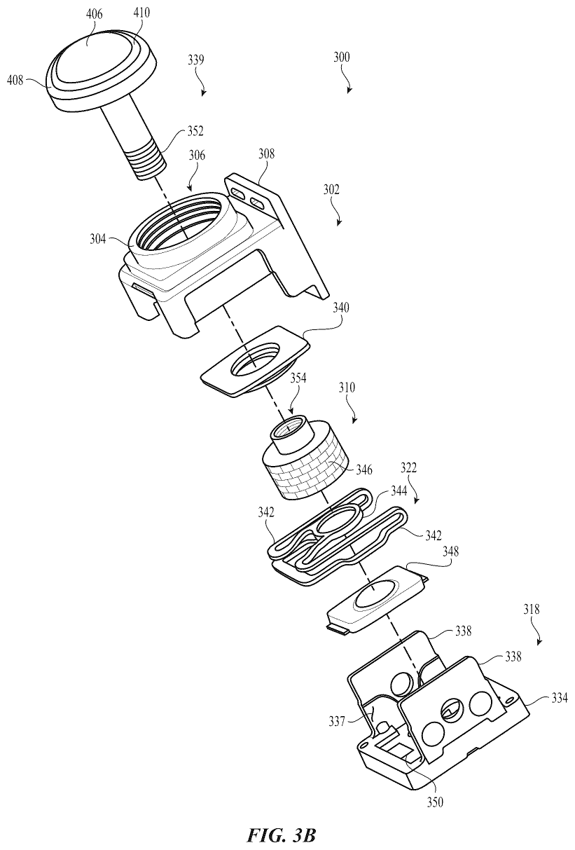

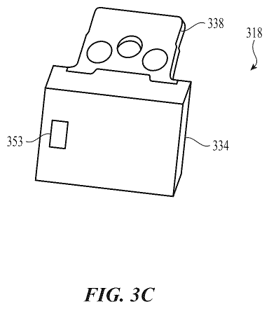

FIG. 3A depicts a sub-assembly 300 of a crown input system, showing components in an assembled configuration. The sub-assembly 300 may include a frame 302 and a bracket 318 (which may be an embodiment of the bracket 218, described with respect to FIG. 2). Together, the frame 302 and the bracket 318 may at least partially enclose a portion of a crown assembly. For example, as shown in FIG. 3A, an optical encoder component 310, which may be attached to a crown shaft, may be positioned within an internal volume defined by the frame 302 and the bracket 318.

The sub-assembly 300 may also include a friction guard 322 (which may be an embodiment of the friction guard 222) and an optical encoder component 310 (which may be an embodiment of the optical encoder component 210). The bracket 318, friction guard 322, and optical encoder component 310 may include the same or similar components and may provide the same or similar functions as the corresponding components described with respect to FIG. 2. Accordingly, details of the components described with respect to FIG. 2 may apply equally to those in FIG. 3A, and for brevity will not be repeated here.

In some cases the portion of the input system shown in FIG. 3A may be a self-contained subassembly. For example, the components shown in FIG. 3A may be assembled together separately from other components, and then integrated with other components of the device, such as a housing, an actuation member, and the like. In this way, components that require tight tolerances and high assembly precision (e.g., the friction guard, the barrel, the frame, and the switch) may be pre-assembled into a secure sub-assembly 300 that can then be incorporated into a device without requiring separate alignment or re-alignment of the components of the sub-assembly 300.

The frame 302 may be attached to a housing of a device, for example via a mounting portion 308. The mounting portion 308 may include openings for receiving fasteners that secure the frame 302 to the housing (e.g., screws, bolts, posts, rivets, welds, stakes, or the like). In some cases, the frame 302 is instead or additionally secured to the housing using adhesives, anchor structures, or any other suitable coupling technique or component. The frame 302 may be formed from or include any suitable material, such as metal, metal alloy, polymer, carbon fiber, or the like. The frame 302 may be a monolithic component, or it may comprise multiple different components that are attached together.

The frame 302 may define an opening 306 that is configured to receive a locking member (e.g., a locking member 404, FIG. 4). The locking member may engage threads in the opening 306 and may capture a portion of a side wall of a housing between a mounting face 304 of the frame 302 and the locking member. An example arrangement of a frame, locking member, and side wall of a housing is shown in FIG. 4.

The bracket 318 may include a body structure 334 (which may be an embodiment of the body structure 234, FIG. 2), and a support structure 336. The body structure 334 may be formed of a polymer material, and may at least partially encapsulate the support structure 336, which may be formed of or include a metal. For example, the support structure 336 may be inserted into a mold cavity, and a polymer or other moldable material may be injected into the mold cavity to partially encapsulate the support structure 336.

The bracket 318 may be attached to the frame 302. For example, the support structure 336 may define flanges 338 that overlap and are secured to a portion of the frame 302. The flanges 338, which may be metal, may be secured to the portion of the frame 302 via any suitable technique, such as welding, brazing, soldering, adhesive, fasteners, or the like. In some cases, the bracket 318 and the frame 302 are carefully aligned, prior to being secured together, to establish the proper alignment and tolerances of the components within the assembly. For example, the positioning of the frame 302 relative to the bracket may define the inner dimension of the assembly, which may define parameters such as the biasing force applied by the friction guard 322 on the optical encoder component 310, the distance between the friction guard 322 and the switch (e.g., the switch 216, FIG. 2), and so forth. Once the frame 302 and the bracket 318 are satisfactorily aligned, they may be secured together (e.g., via welds along the flanges 338, which may be formed of or include metal).

FIG. 3B is an exploded view of the sub-assembly 300, illustrating additional details of the various components and their arrangement. FIG. 3B includes an actuation member 339 (which may be an embodiment of the actuation member 204, FIG. 2). The actuation member 339 may include a threaded region 352 that engages a threaded opening 354 of the optical encoder component 310. The actuation member 339 may be threaded to the optical encoder component 310 after the optical encoder component 310 is assembled into the configuration shown in FIG. 3A.

The sub-assembly 300 may also include a wear plate 340 that is positioned between an inner surface of the frame 302 and a surface of the optical encoder component 310. The wear plate 340 may be configured to reduce friction on the frame 302 due to the rotation of the optical encoder component 310 during rotational inputs to the actuation member 339. The wear plate 340 may also electrically isolate the optical encoder component 310 from the frame 302. More particularly, as noted above, the optical encoder component 310 may define part of the conductive path between the actuation member 339 and biometric sensing circuitry. Thus, the optical encoder component 310 may need to be electrically isolated from conductive components that do not define the conductive path (e.g., components that may be grounded or otherwise interfere with the propagation of signals along the conductive path). Accordingly, the wear plate 340 may be formed of or include an insulating material. In one example construction, the wear plate 340 includes a first metal layer that contacts the optical encoder component 310, a second metal layer that contacts (and is optionally welded to) an inner surface of the frame 302, and an insulating layer between the first and second metal layers. The first and second metal layers may be secured to the insulating layer (which may be a plastic, rubber, foam, or any other suitable material) by adhesive (e.g., a pressure-sensitive adhesive (PSA), heat-sensitive adhesive (HSA), epoxy, cyanoacrylate, or any other suitable adhesive). FIG. 4 shows an example cross-section of a wear plate having multiple layers as described herein.

FIG. 3B also shows additional details of the friction guard 322 and the bracket 318 from FIG. 3A. For example, the friction guard 322 includes a shear plate 344 (which may be an embodiment of the shear plate 226, FIG. 2) and support legs 342 (which may be embodiments of the support legs 224, FIG. 2). The support legs 342 may be flexible and/or deformable, and may act as spring members that support the shear plate 344 and apply a biasing force against the optical encoder component 310 when the sub-assembly 320 is assembled. The support legs 342 may contact a contact portion 350 of a conductor in the bracket 318 to further define the conductive path to the sensing component. The friction guard 322 may be secured to the bracket 318 via adhesive, welding, soldering, brazing, mechanical interlocks, heat staking, or the like. For example, the support legs 342 may be soldered to the contact portion 350 of the bracket 318. The friction guard 322 may also be configured so that the shear plate 344 does not contact the top of the switch 348 when the crown is in an unactuated state (e.g., it is in a rest position and is not being actuated axially). This may help prevent unnecessary wear on the switch 348 and the other components of the crown input system.

The body structure 334 of the bracket 318, which may be formed of an electrical insulator such as a polymer, may extend at least part way up the flanges 338, as shown in FIG. 3B. This extended portion 337 of the body structure 334 may be adjacent the friction guard 322, thus ensuring electrical isolation between the friction guard 322 and the conductive (e.g., metal) support structure 336. The body structure 334 of the bracket 318 may also electrically isolate the flanges 338 (and/or other conductive portions of the bracket 318) from a conductor that is at least partially encapsulated in the body structure 334 and that defines the contact portions 350, 353 (e.g., the conductor 229 or an embodiment thereof). More particularly, the body structure 334 may electrically insulate the conductor from other metallic or conductive components that may otherwise ground or otherwise disrupt the conductive path through the crown input system.

FIG. 3C shows an underside view of the bracket 318 of FIGS. 3A-3B. As described above, the bracket 318 may include a conductor (e.g., the conductor 229, FIG. 2) that is at least partially encapsulated in the body structure 334. As shown in FIG. 3C, a portion of the conductor may be exposed along an exterior surface of the bracket 318 to define an exterior contact portion 353. The exterior contact portion 353 may be conductively coupled to biometric sensing circuitry. More particularly, the exterior contact portion 353 may provide convenient access to the conductive path of the crown, which may otherwise be difficult to physically access as many of the components that define the conductive path may be at least partially encased and/or enclosed in other components (e.g., the frame 302, the bracket 318, etc.) and thus may be otherwise difficult to access in order to complete the conductive path to the biometric sensing circuitry. The contact portion 350 (FIG. 3B) and the exterior contact portion 353 (FIG. 3C) may be portions of a single conductor, such as a metal member.

FIG. 4 is a cross-sectional view of a crown input system installed in a housing. For example, FIG. 4 shows the components of the sub-assembly 300, shown in FIGS. 3A-3B, in a housing defining a side wall 402. The cross-sectional view may correspond to a view through line A-A in FIG. 1.

FIG. 4 illustrates how a mounting face 304 of the frame 302 contacts an inner surface of the side wall 402 of the housing, and a locking member 404 engages with the threads of the frame 302 to capture and/or compress the side wall 402 between the mounting face 304 and a flange of the locking member 404. A seal 412 may be positioned at an interface between the locking member 404 and the side wall 402 to help prevent ingress of water, dust, debris, or other contaminants into the internal volume of the device.

FIG. 4 also illustrates an example crown assembly that is formed from multiple discrete components. For example, the crown assembly may include the optical encoder component 310 and an actuation member that includes a crown shaft 406 and a crown ring 408 coupled to the crown shaft and electrically isolated from the crown shaft (e.g., by an electrical insulating assembly 410, which may be formed from a polymer or other suitable insulating material(s)). In this example, the crown ring 408 may not define part of the conductive path through the crown assembly, and instead the axial end surface of the crown shaft 406 (which is external to the housing and therefore accessible by a user) may define the input surface for the biometric sensor. The actuation member may be coupled to the optical encoder component 310 via threads on the crown shaft 406, as described herein.

The crown ring 408 may include tactile features such as grooves, splines, ridges, textures, or the like, formed on an exterior surface of the crown ring 408 (e.g., the portion of the crown ring that a user touches when providing rotational inputs). The tactile features may be positioned around a circumference of the crown ring 408. For example, the tactile features may include a series of tactile features (e.g., grooves, channels) spaced regularly around a circumferential surface of the crown ring 408. The tactile features may improve a tactile feel of the crown during rotational inputs. For example, the tactile features may provide greater grip or friction between the crown ring 408 and a user's finger, as compared to a smooth or un-featured crown ring 408.

The crown shaft 406 may be electrically isolated from the locking member 404 via one or more bushings 413. The bushings 413 may be formed from a rubber, polymer, or other electrically insulating material. The bushings 413 may also act as guides for the crown shaft 406 to provide smooth operation and maintain alignment, and also to seal the assembly and prevent ingress of water or other contaminants.

FIG. 4 also shows the overlap between the frame 302 and the flanges 338, where the flanges 338 may be affixed to the frame 302. Also, FIG. 4 shows how a three-layer wear plate 340 may be positioned between an inner surface of the frame 302 and the optical encoder component 310 to prevent frictional contact between the optical encoder component 310 and the frame 302 and to maintain electrical separation between the frame 302 and the optical encoder component 310. FIG. 4 further shows an electrically conductive lubricant 414 (e.g., conductive grease) applied at the interface between the shear plate 344 of the friction guard and the end of the optical encoder component 310.

FIG. 5 depicts an example schematic diagram of an electronic device 500. By way of example, the device 500 of FIG. 5 may correspond to the wearable electronic device 100 shown in FIG. 1 (or any other wearable electronic device described herein). To the extent that multiple functionalities, operations, and structures are disclosed as being part of, incorporated into, or performed by the device 500, it should be understood that various embodiments may omit any or all such described functionalities, operations, and structures. Thus, different embodiments of the device 500 may have some, none, or all of the various capabilities, apparatuses, physical features, modes, and operating parameters discussed herein.

As shown in FIG. 5, a device 500 includes a processing unit 502 operatively connected to computer memory 504 and/or computer-readable media 506. The processing unit 502 may be operatively connected to the memory 504 and computer-readable media 506 components via an electronic bus or bridge. The processing unit 502 may include one or more computer processors or microcontrollers that are configured to perform operations in response to computer-readable instructions. The processing unit 502 may include the central processing unit (CPU) of the device. Additionally or alternatively, the processing unit 502 may include other processors within the device including application specific integrated chips (ASIC) and other microcontroller devices.

The memory 504 may include a variety of types of non-transitory computer-readable storage media, including, for example, read access memory (RAM), read-only memory (ROM), erasable programmable memory (e.g., EPROM and EEPROM), or flash memory. The memory 504 is configured to store computer-readable instructions, sensor values, and other persistent software elements. Computer-readable media 506 also includes a variety of types of non-transitory computer-readable storage media including, for example, a hard-drive storage device, a solid-state storage device, a portable magnetic storage device, or other similar device. The computer-readable media 506 may also be configured to store computer-readable instructions, sensor values, and other persistent software elements.

In this example, the processing unit 502 is operable to read computer-readable instructions stored on the memory 504 and/or computer-readable media 506. The computer-readable instructions may adapt the processing unit 502 to perform the operations or functions described above with respect to FIGS. 1-4. In particular, the processing unit 502, the memory 504, and/or the computer-readable media 506 may be configured to cooperate with a sensor 524 (e.g., a rotation sensor that senses rotation of a crown component or a sensor that senses motion of a user's finger) to control the operation of a device in response to an input applied to a crown of a device (e.g., the crown 112). The computer-readable instructions may be provided as a computer-program product, software application, or the like.

As shown in FIG. 5, the device 500 also includes a display 508. The display 508 may include a liquid-crystal display (LCD), organic light emitting diode (OLED) display, light emitting diode (LED) display, or the like. If the display 508 is an LCD, the display 508 may also include a backlight component that can be controlled to provide variable levels of display brightness. If the display 508 is an OLED or LED type display, the brightness of the display 508 may be controlled by modifying the electrical signals that are provided to display elements. The display 508 may correspond to any of the displays shown or described herein.

The device 500 may also include a battery 509 that is configured to provide electrical power to the components of the device 500. The battery 509 may include one or more power storage cells that are linked together to provide an internal supply of electrical power. The battery 509 may be operatively coupled to power management circuitry that is configured to provide appropriate voltage and power levels for individual components or groups of components within the device 500. The battery 509, via power management circuitry, may be configured to receive power from an external source, such as an AC power outlet. The battery 509 may store received power so that the device 500 may operate without connection to an external power source for an extended period of time, which may range from several hours to several days.

In some embodiments, the device 500 includes one or more input devices 510. An input device 510 is a device that is configured to receive user input. The one or more input devices 510 may include, for example, a crown input system, a push button, a touch-activated button, a keyboard, a keypad, or the like (including any combination of these or other components). In some embodiments, the input device 510 may provide a dedicated or primary function, including, for example, a power button, volume buttons, home buttons, scroll wheels, and camera buttons.

The device 500 may also include a sensor 524. The sensor 524 may detect inputs provided by a user to a crown of the device (e.g., the crown 112). As described above, the sensor 524 may include sensing circuitry and other sensing components that facilitate sensing of rotational motion of a crown, as well as sensing circuitry and other sensing components (optionally including a switch) that facilitate sensing of axial motion of the crown. The sensor 524 may include components such as an optical detector (e.g., the optical detector 212), a barrel (e.g., the barrels 210, 310), a tactile or dome switch (e.g., the switches 216, 348), or any other suitable components or sensors that may be used to provide the sensing functions described herein. The sensor 524 may also be a biometric sensor, such as a heart rate sensor, electrocardiograph sensor, temperature sensor, or any other sensor that conductively couples to user and/or to the external environment through a crown input system, as described herein. In cases where the sensor 524 is a biometric sensor, it may include biometric sensing circuitry, as well as portions of a crown that conductively couple a user's body to the biometric sensing circuitry. Biometric sensing circuitry may include components such as processors, capacitors, inductors, transistors, analog-to-digital converters, or the like.

The device 500 may also include a touch sensor 520 that is configured to determine a location of a touch on a touch-sensitive surface of the device 500 (e.g., an input surface defined by the portion of a cover 108 over a display 109). The touch sensor 520 may use or include capacitive sensors, resistive sensors, surface acoustic wave sensors, piezoelectric sensors, strain gauges, or the like. In some cases the touch sensor 520 associated with a touch-sensitive surface of the device 500 may include a capacitive array of electrodes or nodes that operate in accordance with a mutual-capacitance or self-capacitance scheme. The touch sensor 520 may be integrated with one or more layers of a display stack (e.g., the display 109) to provide the touch-sensing functionality of a touchscreen. Moreover, the touch sensor 520, or a portion thereof, may be used to sense motion of a user's finger as it slides along a surface of a crown, as described herein.

The device 500 may also include a force sensor 522 that is configured to receive and/or detect force inputs applied to a user input surface of the device 500 (e.g., the display 109). The force sensor 522 may use or include capacitive sensors, resistive sensors, surface acoustic wave sensors, piezoelectric sensors, strain gauges, or the like. In some cases, the force sensor 522 may include or be coupled to capacitive sensing elements that facilitate the detection of changes in relative positions of the components of the force sensor (e.g., deflections caused by a force input). The force sensor 522 may be integrated with one or more layers of a display stack (e.g., the display 109) to provide force-sensing functionality of a touchscreen.

The device 500 may also include a communication port 528 that is configured to transmit and/or receive signals or electrical communication from an external or separate device. The communication port 528 may be configured to couple to an external device via a cable, adaptor, or other type of electrical connector. In some embodiments, the communication port 528 may be used to couple the device 500 to an accessory, including a dock or case, a stylus or other input device, smart cover, smart stand, keyboard, or other device configured to send and/or receive electrical signals.

As described above, one aspect of the present technology is the gathering and use of data available from a user. The present disclosure contemplates that in some instances, this gathered data may include personal information data that uniquely identifies or can be used to contact or locate a specific person. Such personal information data can include demographic data, location-based data, telephone numbers, email addresses, twitter ID's, home addresses, data or records relating to a user's health or level of fitness (e.g., vital signs measurements, medication information, exercise information), date of birth, or any other identifying or personal information. For example, where the sensor described above is a biometric sensor, sensitive and/or personal information may be captured about a user (e.g., health-related data).

The present disclosure recognizes that the use of such personal information data, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide mood-associated data for targeted content delivery services. In yet another example, users can select to limit the length of time mood-associated data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings. Also, when used herein to refer to positions of components, the terms above and below, or their synonyms, do not necessarily refer to an absolute position relative to an external reference, but instead refer to the relative position of components with reference to the figures.

What is claimed is:

1. An electronic watch comprising:
a housing defining a side wall having a through-hole;
a crown assembly comprising:
an actuation member comprising:
a crown shaft extending through the through-hole and having an exterior portion defining an input surface; and
a crown ring coupled to the exterior portion of the crown shaft and electrically isolated from the crown shaft; and
an optical encoder component attached to the actuation member and defining a group of optical features;
an optical detector configured to detect rotation of the crown assembly by detecting motion of the group of optical features; and
an electrocardiogramansor comprising a sensing component; wherein
the sensing component is conductively coupled to the actuation member via a conductive path at least partially defined by the crown shaft.

2. The electronic watch of claim 1, wherein:
the electronic watch further comprises:
a switch configured to be actuated by the crown assembly in response to an axial input applied to the crown assembly;
a friction guard having a shear plate positioned between the switch and the optical encoder component and conductively coupled to the optical encoder component; and
a bracket comprising:
a body structure; and
a conductor coupled to the body structure; and
the conductive path is further defined by the optical encoder component and the conductor and is configured to carry electrical signals from a user in contact with the input surface to the electrocardiogramansor.

3. The electronic watch of claim 2, wherein:
the body structure comprises a polymer material; and
the conductor is at least partially encapsulated by the polymer material.

4. The electronic watch of claim 1, wherein:
the electronic watch further comprises:
a switch configured to be actuated by the crown assembly in response to an axial force applied to the crown assembly; and
a friction guard at least partially positioned between the switch and the optical encoder component and conductively coupled to the optical encoder component;
the axial force is transferred from the crown assembly to the switch through the friction guard; and
the conductive path is further defined by the friction guard.

5. The electronic watch of claim 4, wherein:
the crown assembly is rotatable relative to the housing;
the crown assembly rotates against the friction guard when the crown assembly is rotated; and
the friction guard protects the switch from rotational friction from the crown assembly.

6. The electronic watch of claim 1, further comprising, within an internal volume of the housing:
a bracket;
a frame coupled to the bracket and attached to the housing; and
a wear plate positioned between an inner surface of the frame and configured to electrically isolate the optical encoder component from the frame.

7. The electronic watch of claim 1, wherein:
the electronic watch further comprises:
a display positioned at least partially within the housing and configured to display a graphical output;
a transparent cover coupled to the housing; and
a touch sensor positioned below the transparent cover and configured to detect touch inputs applied to the transparent cover; and
the electronic watch is configured to modify the graphical output in accordance with a detected rotation of the crown assembly.

8. An electronic watch comprising:
a housing at least partially defining an internal volume;
biometric sensing circuitry within the internal volume; and
a crown assembly configured to rotate and translate relative to the housing and comprising:
a crown shaft defining an axial end surface;
a crown ring coupled to the crown shaft and defining a series of tactile features arranged around a circumference of the crown ring; and
an optical encoder component attached to the crown shaft and defining a group of optical features, wherein
the crown shaft and the optical encoder component define a conductive path configured to conductively couple the crown assembly to the biometric sensing circuitry.

9. The electronic watch of claim 8, wherein the biometric sensing circuitry is part of an electrocardiograph sensor.

10. The electronic watch of claim 8, further comprising:
a switch; and
a friction guard having a shear plate positioned between the crown assembly and the switch and configured to transfer axial forces from the crown assembly to the switch.

11. The electronic watch of claim 10, wherein:
the electronic watch further comprises:
a bracket positioned in the internal volume and comprising:
a metal flange;
a polymer body structure; and
a conductor coupled to the polymer body structure and electrically isolated from the metal flange by the polymer body structure;
the friction guard is in contact with the conductor; and
the conductive path is further defined by the friction guard and the conductor.

12. The electronic watch of claim 10, further comprising a conductive lubricant at an interface between the crown assembly and the friction guard.

13. The electronic watch of claim 8, wherein the optical encoder component is attached to the crown shaft via a threaded connection.

14. The electronic watch of claim 8, wherein the axial end surface of the crown shaft defines an input surface for the biometric sensing circuitry.

15. An electronic device comprising:
a housing at least partially defining an internal volume;
an electrocardiograph sensor within the internal volume and comprising a sensing component;
a frame within the internal volume and attached to the housing;
a bracket attached to the frame and comprising:
a body structure; and
a conductor at least partially encapsulated in the body structure;
a crown assembly having an external portion positioned outside of the internal volume and configured to rotate and translate relative to the housing, wherein
the bracket and the frame at least partially enclose a portion of the crown assembly; and
the crown assembly and the conductor define a conductive path configured to conductively couple the sensing component to an object in contact with the external portion of the crown assembly.

16. The electronic device of claim 15, further comprising:
a band coupled to the housing;
a display at least partially within the internal volume of the housing;
a transparent cover coupled to the housing and positioned such that the display is visible through the transparent cover; and
a touch sensor at least partially within the internal volume and configured to detect touch inputs applied to the transparent cover.

17. The electronic device of claim 15, wherein:
the crown assembly comprises:
a crown shaft having an internal portion within the internal volume; and
an optical encoder component attached to the internal portion of the crown shaft and defining a group of optical features;
the electronic device further comprises an optical detector configured to detect rotation of the crown assembly by detecting motion of the group of optical features; and
the optical encoder component defines part of the conductive path.

18. The electronic device of claim 15, further comprising:
a friction guard in contact with the bracket and the crown assembly; and
a switch positioned between the friction guard and the bracket and configured to be actuated in response to axial translation of the crown assembly.

19. The electronic device of claim 18, wherein the friction guard comprises:
a shear plate; and
a support leg configured to allow the shear plate to deflect relative to the bracket.

20. The electronic device of claim 19, wherein the friction guard is a single piece of conductive material.
